(19) United States
(12) Patent Application Publication
(10) Pub. No.: US 2025/0213816 A1
(43) Pub. Date: Jul. 3, 2025

(54) REEL CARTRIDGE AND CARTRIDGE HOUSING

(71) Applicant: Reece BLACKWOOD, Inman, SC (US)

(72) Inventor: Reece BLACKWOOD, Inman, SC (US)

(21) Appl. No.: 19/004,250

(22) Filed: Dec. 28, 2024

Related U.S. Application Data
(60) Provisional application No. 63/616,125, filed on Dec. 29, 2023.

(51) Int. Cl. A61M 25/00; A61M 25/09; B65D 25/04; B65D 25/28; B65D 81/22; B65D 83/00; B65D 85/04; B65H 75/44

(52) U.S. Cl. CPC A61M 25/002; A61M 25/09; B65D 25/04; B65D 25/2826; B65D 81/22; B65D 83/00; B65D 85/04; B65H 75/4471

(57) ABSTRACT

An organization system may include a slotted housing comprising a top notch. An organization system may include a cartridge configured to receive a spooled elongated profile object, and configured to be received by a slot of the slotted housing, wherein, the top notch is defined by a housing wall, wherein the slot and the housing wall form a soaking bath for the cartridge and the spooled elongated profile object therein; and wherein, when the spooled elongated profile object is unspooled from the cartridge, the top notch limits the cartridge from rocking or rotating within the slot.

### BACKGROUND

The present disclosure relates to reinforced polymeric based multi-material geometrically enhanced structural compositions and related methods. More particularly, to polymeric, multi-material, multi-layer, geometric infilled components and methods of making the same.

In the realm of tools, devices, and equipment for surgical, endovascular, radiology, and other invasive or partially invasive procedures (with a specific focus on organizers, dispensers, spools, reels, and holders designed for wires, catheters, sheaths, stents, and similar elongated profile objects), the efficient management of these tools, devices, and equipment is a critical consideration and often a challenge. This challenge is particularly pronounced in endovascular procedures, where a variety of wires, catheters, sheaths, stents, or other elongated profile objects, often of small diameters and extended lengths, are introduced and removed from a patient.

In endovascular procedures, for example, the exchange of wires, catheters, sheaths, stents, or other elongated profile objects, within a patient's arterial or venous access point is commonplace. However, inherent challenges arise due to the diverse characteristics of guidewires, catheters, sheaths, stents, and comparable objects, including variations in size, length, gauge, and inner and outer diameters. The potential for disorganization, confusion, and breaches in sterility protocols is particularly pronounced during surgeries involving guidewires and catheters.

The absence of a systematic organization approach and appropriate tools intensifies these challenges, posing risks such as inadvertent removal of guidewires, use of incorrect catheters, and disruptions in sterility that may lead to complications like clotting or infection. Even skilled surgical professionals face difficulties in managing the diverse array of wires, catheters, sheaths, stents, and similar elongated profile objects, underscoring the need for a cohesive organization plan and tools to mitigate the risk of procedural failure.

For example, endovascular procedures and interventional radiology sometimes demand tools, devices, and equipment that even skilled surgical technicians are unfamiliar with. Sometimes, the incorrect tool, device, and equipment is passed during the procedure, resulting in delays or negative consequences. Sometimes, these mistakes can lead to surgical failure.

The systems and methods according to the present disclosure directly addresses the real-world challenges in the field, particularly in situations where sterility and rapid access to necessary tools are crucial, such as emergencies or trauma scenarios. The organization tools and related methods described herein serve to effectively organize, make available, label, and categorize each wire, catheter, sheath, stent, or similar elongated profile object used during a procedure. These systems and methods stand to deliver tangible benefits, including a reduction in incorrect handoffs, improved ergonomics of tools and equipment, enhanced communication among professionals involved in a procedure, and, ultimately, the promotion of more effective, safe, and efficient surgical, endovascular, radiological, and other invasive procedures.

### SUMMARY

In some aspects, the techniques described herein relate to an organization tool or device, and associated methods, involving cartridges and slotted housing.

In some aspects, the techniques described herein relate to a cartridge with an entry/exit port, or with a port with channel, that can be entered by pushing a wire, a catheter, a sheath, a stent, and/or other elongated profile object in through the port, or by placing the wire, the catheter, and/or the other elongated profile object in from the side, using the channel. The port with channel may have a “T” design that facilitates a side-entry and the facilitates dispensing/loading, but that prevents unwanted movement from the cartridge.

In some aspects, the techniques described herein relate to a housing and cartridge(s) designed to house, clean, receive, dispense, label, and/or organize wire(s), catheter(s), or other elongated profile object(s) used during surgical, endovascular, radiology, and other invasive or partially invasive procedures.

In some aspects, the techniques described herein relate to a housing that neatly holds one or more reel cartridges or “cartridges” (as used herein for both reel cartridges and non-mechanical cartridges with no moving parts) (in one aspect, 6 total cartridges) in a front facing direction, for ease of use, and for streamlining the handoff and take-back of guidewire(s) and catheter(s) from the cartridge(s). The top notch of each slot (in one aspect, a notch on a wall defining a slot) of the housing prevents the corresponding cartridge for that slot from rocking or rotating within the housing.

In some aspects, the housing defines separate, individual “baths” or slots for each cartridge such that wires and/or catheters held with the cartridge can be soaked in heparinized saline or water, or any other liquid or solution called for by surgical, endovascular, radiology, and other invasive or partially invasive procedures. The slots have solid walls on either side so that less solution is used for each slot and maximizing contact between solution and guidewire/catheter.

In some aspects, when an inner disk of a reel cartridge (described in greater detail herein) spins in a bath, the guidewire or the catheter spooled within the cartridge can rotate in space within a slot such that, if the slot is full of heparinized saline, or any other liquid or solution, the guidewire or the catheter will rotate and be cleaned within the bath.

In some aspects, the techniques described herein relate to a cartridge that includes three parts or components. The cartridge holds, spools, and retains a guidewire or catheter securely and in an organized manner. The cartridge consists of an outer disk, an inner rotating disk, and a feeder at the top of the outer disk with a small handle that can be grabbed with two fingers. The handle allows for precision and speed when loading and unloading the cartridges into a housing or into a separate holder/receiver/cleaner. The inner disk rotates inside the outer disk (a disk within disk configuration) to allow the tension of the wire and/or catheter (acting on a surface within the cartridge, for example) to not alter the speed at which loading can be performed, and therefore allows for a smooth-feeling loading and unloading action (e.g., reduces friction between the wire or catheter and cartridge). Labeling can be done either by applying a sterile sticky label on the slanted section of the cartridge body to allow easy viewing, or using a sterile marker and writing directly on the slanted section of the cartridge body. Labeling can also be done either by applying a sterile sticky label on the slanted section of the feeder to allow easy viewing, or using a sterile marker and writing directly on the slanted section of the feeder.

In some aspects, the techniques described herein relate to a method for loading the guidewire or catheter into a cartridge, wherein an assembled cartridge has a feeder removed from the assembled cartridge body, such that a pre-coiled or pre-spooled guidewire or catheter can be placed into a receptacle space of the cartridge body, such that an end of the guidewire or catheter can be threaded through an opening defined by the feeder, and such that the end of the guidewire or catheter can be accessed from outside the feeder when the cartridge is fully assembled again.

In some aspects, the techniques described herein relate to another method for loading the guidewire or catheter into a cartridge, wherein a fully assembled cartridge has an end of a guidewire or catheter threaded into and through an opening defined by the feeder into a assembled cartridge, such that the guidewire or catheter can be pushed into a receptacle space of a cartridge body of the assembled cartridge (to coil or spool the guidewire or catheter within the receptacle space), and such that an opposite end of the guidewire or catheter is accessible through the feeder opening (to dispense the guidewire or catheter from the assembled cartridge. The opening defined by the feeder may be configured as a port with channel, wherein the channel is angled towards an inner disc of the assembled cartridge.

In some aspects, the techniques described herein relate to a method of using a reel cartridge and cartridge housing including: loading heparinized saline, or any other liquid or solution, into a slot(s) of the housing; placing a pre-loaded cartridge(s) in the slot(s) bath; dispensing and/or reloading the guidewire or catheter from the cartridge(s); if reloading used-guidewire or used-catheter, allowing the reloaded guidewire or reloaded catheter to coil or spool within the cartridge such that the bath can clear the guidewire or catheter of blood clots, for example; and visually labeling the cartridge(s) on the surface(s) visible when in the cartridge is in the housing, such that users can clearly see the “LABEL”(s) from any angle or from a broad vantage point. As new guidewires or new catheters are requested, another cartridge(s) can be loaded with the guidewire or catheter. During use, the guidewire and the catheter can be dispensed from the cartridge while the cartridge is in the housing or by removing the cartridge from the housing and passing the cartridge.

In some aspects, the techniques described herein relate to a reel cartridge and cartridge housing or to an organization tool or device or system that has nothing to do with surgical, endovascular, radiology, and other invasive or partially invasive procedures. Instead, the reel cartridge and cartridge housing, or the organization tool or device or system, may be directed to various other fields such as: systems and methods for fishing, systems and methods for environmental reclamation and clean up, systems and methods for recycling; systems and methods for threads and clothing; systems and methods for directional drilling; systems and methods oil and gas exploration; systems and methods for submersible and sea/water body exploration; systems and methods for tethers; system and methods for space exploration and low and high orbit.

### BRIEF DESCRIPTION OF THE DRAWINGS

Many aspects of the present disclosure will be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views. It should be recognized that these implementations and embodiments are merely illustrative of the principles of the present disclosure. Therefore, in the drawings:

FIG. 1 is a perspective view of an illustration of an example cartridge according to the present disclosure.

FIG. 2 is a perspective view of an illustration of an example housing according to the present disclosure.

FIG. 3 is a perspective view of an illustration of an example housing with a cartridge in a slot according to the present disclosure.

FIG. 4 is an exploded top perspective view of an illustration of an example cartridge according to the present disclosure.

FIG. 5 is an exploded side perspective view of an illustration of an example cartridge according to the present disclosure.

FIG. 6 is an exploded side elevation view of an illustration of an example cartridge according to the present disclosure.

FIG. 7 is a perspective view of an illustration of an example feeder according to the present disclosure.

FIG. 8 is a perspective view of an illustration of an example feeder according to the present disclosure.

FIG. 9 is a perspective view of an illustration of an example feeder according to the present disclosure.

FIG. 10 is a bottom side elevation view of an illustration of an example feeder according to the present disclosure.

FIG. 11 is a bottom perspective view of an illustration of an example cartridge according to the present disclosure.

FIG. 12 is a cross section view of an illustration of an example reel cartridge with housing according to the present disclosure.

FIG. 13 is a cross section view of an illustration of an example reel cartridge with housing according to the present disclosure.

FIG. 14 is a front view of an illustration of an example reel cartridge with housing according to the present disclosure.

FIG. 15 is a rear view thereof.

FIG. 16 is a left side view thereof.

FIG. 17 is a right side view thereof.

FIG. 18 is a top view thereof.

FIG. 19 is a first side exploded perspective view thereof.

FIG. 20 is a second side exploded perspective view thereof.

FIG. 21 is a first side perspective view of an illustration of an example reel cartridge according to the present disclosure.

FIG. 22 is a second side perspective view thereof.

FIG. 23 is a front view thereof.

FIG. 24 is a rear view thereof.

FIG. 25 is a left side view thereof.

FIG. 26 is a right side view thereof.

FIG. 27 is a top view thereof.

FIG. 28 is a bottom view thereof.

FIG. 29 is a first side perspective view of an illustration of an example reel cartridge according to the present disclosure.

FIG. 30 is a second side perspective view thereof.

FIG. 31 is a third side perspective view thereof.

FIG. 32 is a fourth side perspective view thereof.

FIG. 33 is a front view thereof.

FIG. 34 is a rear view thereof.

FIG. 35 is a left side view thereof.

FIG. 36 is a right side view thereof.

FIG. 37 is a top view thereof.

FIG. 38 is a bottom view thereof.

FIG. 39 is a first side perspective view of an illustration of an example reel cartridge according to the present disclosure.

FIG. 40 is a second side perspective view thereof.

FIG. 41 is a third side perspective view thereof.

FIG. 42 is a fourth side perspective view thereof.

FIG. 43 is a front view thereof.

FIG. 44 is a rear view thereof.

FIG. 45 is a left side view thereof.

FIG. 46 is a right side view thereof.

FIG. 47 is a top view thereof.

FIG. 48 is a bottom view thereof.

### DETAILED DESCRIPTION

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

Throughout this specification and the claims, the terms “comprise,” “comprises”, and “comprising” are used in a non-exclusive sense, except where the context requires otherwise. Likewise, the term “includes” and its grammatical variants are intended to be non-limiting, such that recitation of items in a list is not to the exclusion of other like items that can be substituted or added to the listed items.

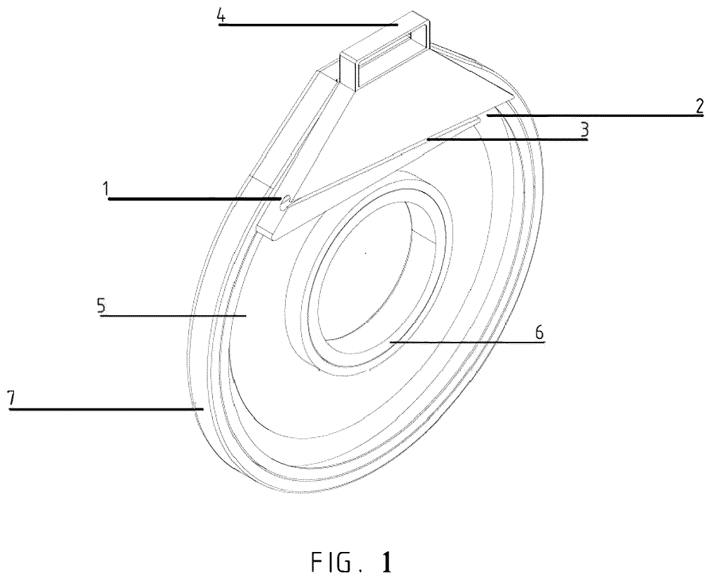

Turning now to the FIGS., FIG. 1 is a perspective view of a Cartridge. The Cartridge (FIG. 1) is used to hold guidewires or catheters by loading them into a hole or a port of a feeder (1). In one aspect, the feeder (1) is a triangular feeder. In another aspect, the feeder (1) is a three dimensional polygon shape with faces that are visible from various different angles and lines of sight. In another aspect, the feeder (1) is in the shape of a stepped pyramid without a top handle, and in such configuration the cartridge body/outer disk (7) or feeder (1) outer surface may have a handling portion, finger groove(s), indent(s), divot(s), and/or surface feature(s) to facilitate handling the cartridge (not shown). Moreover, in one aspect, when the Cartridge is in the housing (see FIG. C) the handle (4) aids in removing the cartridge from the housing during operation or disposal.

When loading the Cartridge with a guidewire(s) or a catheter(s), for example, from the outside into the cartridge, via the hole or port in the feeder (1), the inner disk (5) rotates around a raised boss (6) to allow tension on the wire/catheter to be dispersed in the cartridge as the wire/catheter is pushed into the cartridge. In one aspect, the raised boss (6) and the outer disk (7) are a single monolithic component. Moreover, in another aspect, the optional slit (3) in the triangular feeder (1) allows for an alternative way to load the Cartridge with the wire/catheter, for example. The alternative way to load the Cartridge, for example, is to coil the wire/catheter by hand and to set it into the inner disk (5) with an end of the wire/catheter slid into the slit of the feeder (3), from the inside of the cartridge, such that the wire/catheter can be pulled and uncoiled out of the cartridge through the hole or port of the feeder (1). In another aspect, the optional notch (2) at the end of the slit (3) allows for easier placement of the end of the wire/catheter into the feeder (1), when placing a wire/catheter in the inner disk (5) for the alternative method, and/or when smoothly transitioning the wire/catheter into or out of the Cartridge during use or loading.

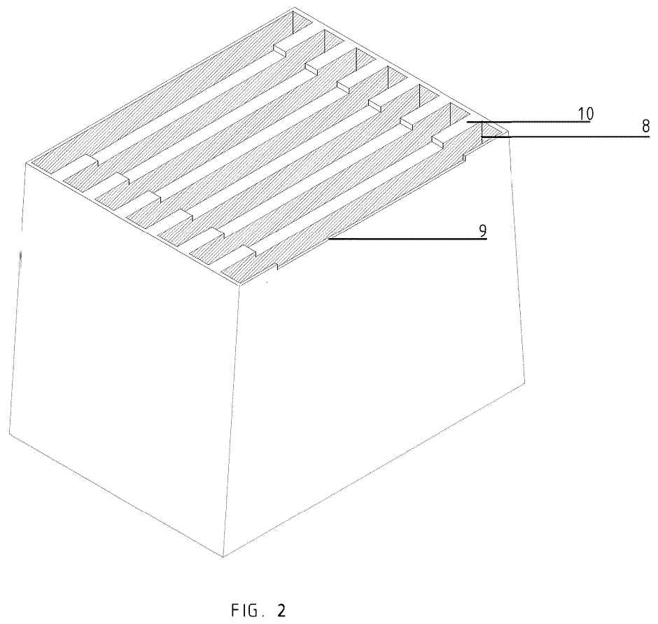

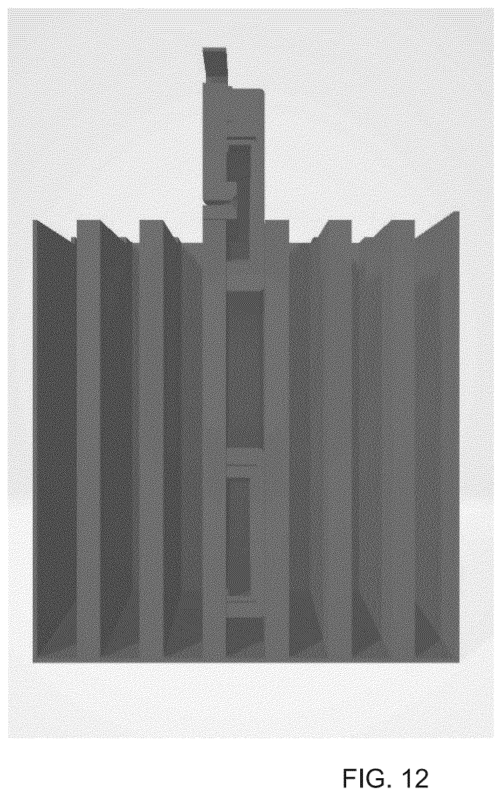

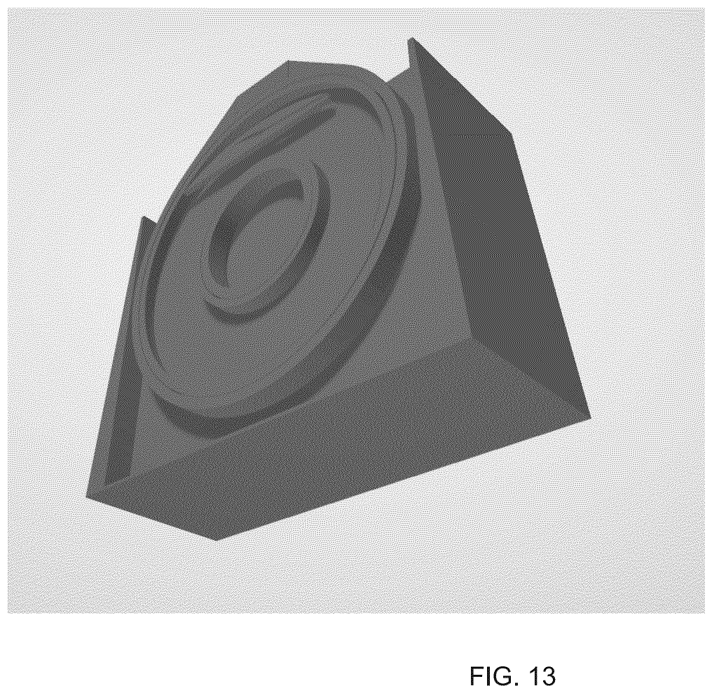

FIG. 2 is a perspective view of a housing. The housing (FIG. 2) has slots (8) for seven Cartridges to be held uniformly in one direction. In one aspect, the housing may have as many slot(s) (8) as are needed, and the slot(s) (8) may hold one or more cartridges. In this way, the housing is not limited to seven slots (8) each for only one of seven cartridges. Moreover, in one aspect, a groove/notch (9) on a wall (10) (the wall (10) defining a portion of the slot (8)) of the housing prevents a corresponding cartridge from rotating inside the housing. Moreover, in another aspect, because the feeder (1) on the Cartridge corresponds to the groove/notch (9), the slot (8) is configured to receive the cartridge only in direction/orientation (e.g., the feeder (1) of the Cartridge must line up with the groove/notch (9) when received by the slot (8). Moreover, in another aspect, the walls (10) of the housing are wide enough for the Cartridge to sit flush with an adjacent Cartridge(s). FIG. 12 and FIG. 13 (which are sectional views of the Cartridge and housing, illustrated as cutting through the feeder (1) and leaving behind a seemingly floating portion of the feeder (1) in space), in particular, show how the Cartridge interfaces with the housing to maximize space in and around the surrounding areas of the housing to create a compact system.

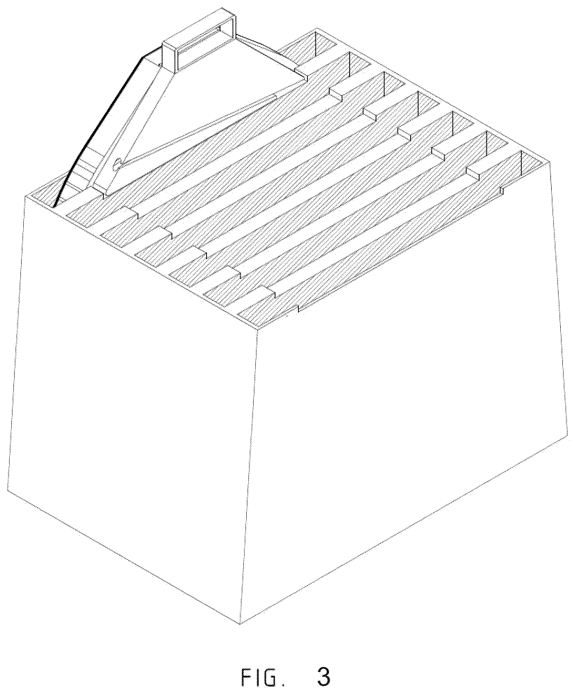

FIG. 3 is a perspective view of a housing with a cartridge in a slot. In one aspect, as illustrated, the Cartridge is oriented in the housing in the only direction it can be received such that the groove (9) can receive the feeder (1) of the Cartridge. As such, in another aspect, the groove (9) eliminates confusion for Cartridge orientation while in use or in storage in the housing. Moreover, in another aspect, when the Cartridge is inserted correctly into the slot (8) of the housing, the movement of the inner disk (5) in the housing (see FIG. 1) starts the cleaning process by moving the wire/catheter around solution that can be placed into the slot (8) and agitating the solution around the wire/catheter.

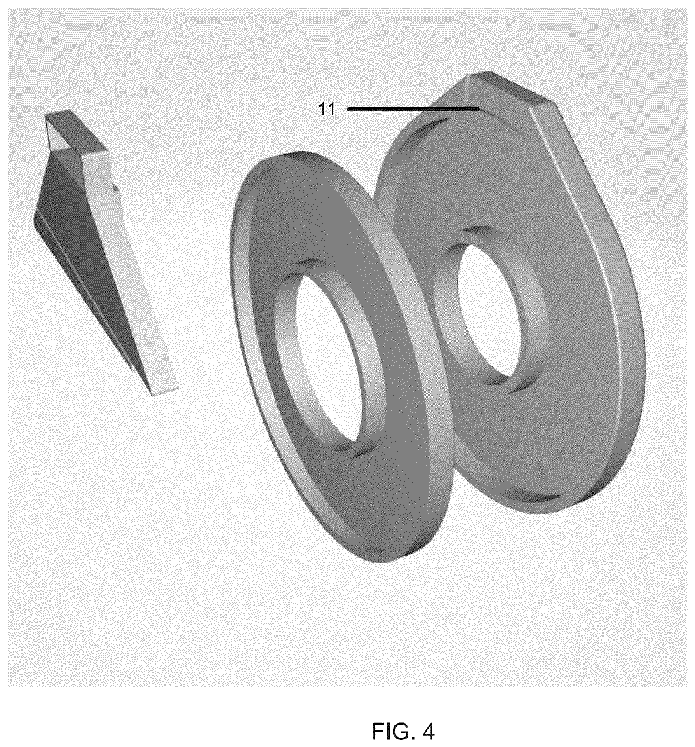

FIG. 4 is an exploded top perspective view of the Cartridge of FIG. 1. Only major differences or points of emphasis are described herein. In particular, this view shows the ledge (11) that the feeder (1) of the Cartridge rests on. In one aspect, the ledge (11) helps with centering and placement of the feeder (1) when mating with the outer disk (7). In another aspect, the ledge (11) is precisely positioned in the top center of the outer disk (7) to allow for proper clearance between the inner disk (5) and the feeder (1), as well as holding the inner disk (5) in its position within the outer disk (7). In another aspect, the inner disk (5) is manufactured with tight tolerance (e.g., 0.5 mm-1 mm, or 0.3 mm- 0.8 mm, or 0.2 mm-0.5 mm) to the raised boss (6) of the outer disk/cartridge body (7) for proper rotation and secure fit.

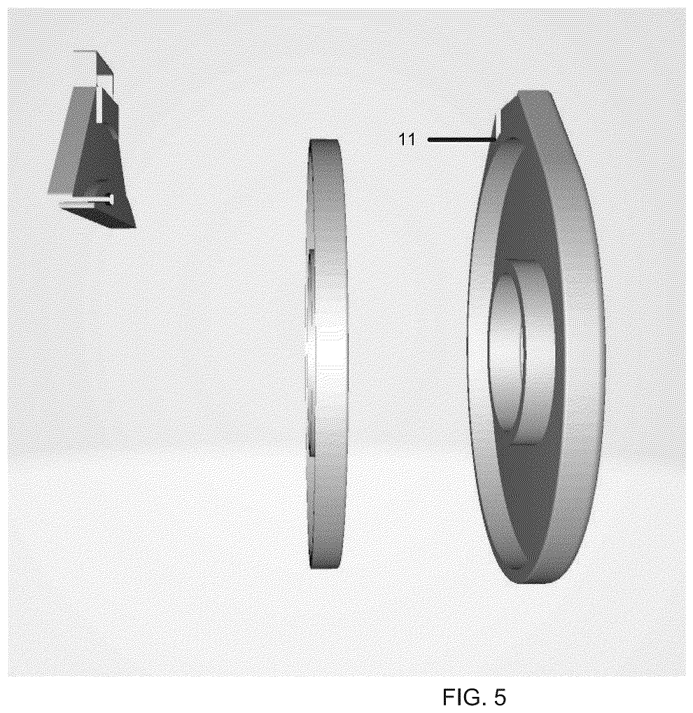

FIG. 5 is an exploded side perspective view of the Cartridge of FIG. 1. Only major differences or points of emphasis are described herein. In particular, this view shows all three component profiles of a Cartridge: a feeder (1); an inner disk (5), and an outer disk/cartridge body (7). Additionally, a feeder hole of the feeder (1) can be seen. In one aspect, the feeder hole is at an angle which, when the Cartridge is fully assembled, allows for wire/catheter going through the feeder hole, into the Cartridge, to be directed towards the inner disk (5). In another aspect, the angle is designed to keep the wire/catheter guided toward the back of the inner disk (5) to reduce chances of an end of the wire/catheter or a portion of coiled/spooled wire/catheter from springing out of the assembled Cartridge. The angle can be in a small range of 4.9 degrees minimum to a maximum of 7 degrees. In another aspect, the angle can be in a small range of 4.7 degrees minimum to a maximum of 7.2 degrees. In another aspect, the angle can be in a small range of 4.5 degrees minimum to a maximum of 7.4 degrees. In another aspect, the angle is 5.39 degrees.

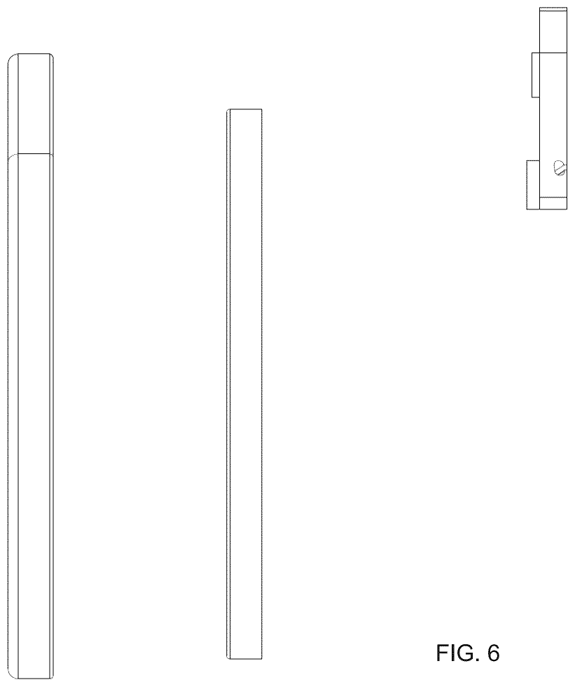

FIG. 6 is an exploded side elevation view of the Cartridge of FIG. 1. Only major differences or points of emphasis are described herein. In particular, this view shows the space where the inner disk sits and rotates. This space is at an exact height that allows for the inner disk (5) to sit flush with the outer disk (7) and to allow seamless rotation with little to no binding issues.

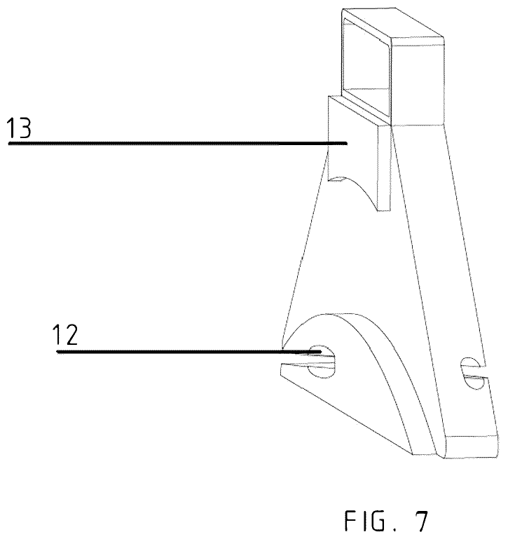

FIG. 7 is a first perspective view of the feeder of FIG. 1. Only major differences or points of emphasis are described herein. In particular, this view shows the back features of the feeder (1) such as: the inner wire/catheter guide (12) at the opposite end of the hole or port of the feeder (1); and an extrusion (13) where the feeder (1) connects or mates with the outer disk/cartridge body (7), for example. In one aspect, the extrusion is arched at its base to more easily level the feeder upon assembly with the outer disk/cartridge body (7). In another aspect, the “arched” shape of the wire guide is intended to help the wire/catheter going through the feeder (1) clear the inner disk (5), and to allow for grip and stabilization for the assembled Cartridge.

FIG. 8 is a second perspective view of the feeder of FIG. 1. Only major differences or points of emphasis are described herein. In particular, this view shows the slit (3) used for side entry for a wire/catheter, for example, or for the alternative loading method(s) as described herein. This view also shows one representation of an angle of the feeder hole and/or of the slit (3) of the feeder (1). Moreover, this view also shows one representation of surface features that can be present on the surface of the wire/catheter guide (12) to allow for sliding and to minimize friction acting on the wire/catheter being pulled or pushed through the feeder (1).

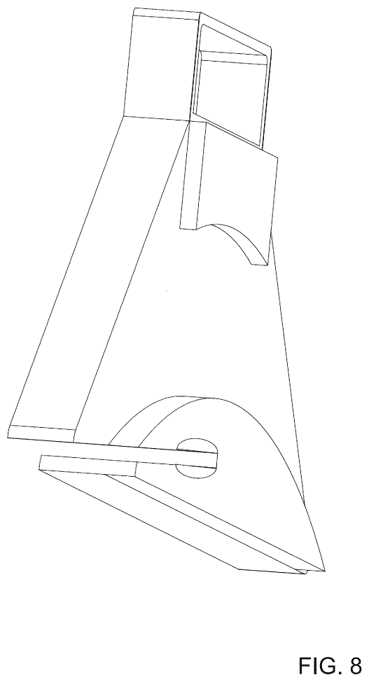

FIG. 9 is a third perspective view of the feeder of FIG. 1. Only major differences or points of emphasis are described herein. In particular, this view shows one representation of an angle of the feeder hole or port of the feeder (1). This view also shows one representation of the slit (3) and how its structure and position relate to the feeder hole or port of the feeder (1).

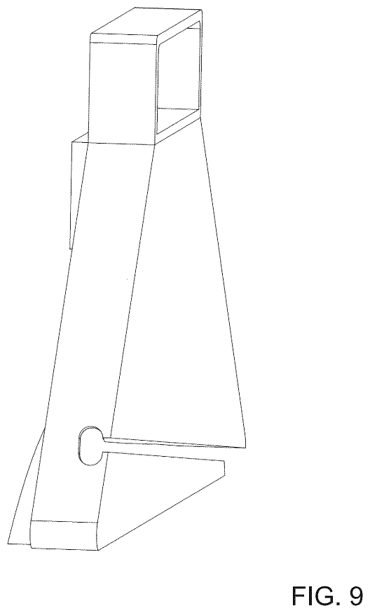

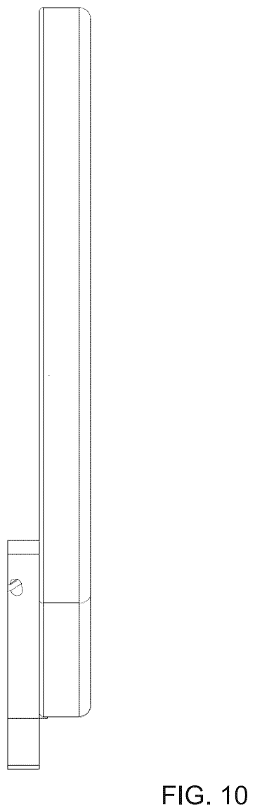

FIG. 10 is an upside down side elevation view of the cartridge of FIG. 1. Only major differences or points of emphasis are described herein. In particular, this view shows one representation of an overall side profile of a Cartridge including a handle (4).

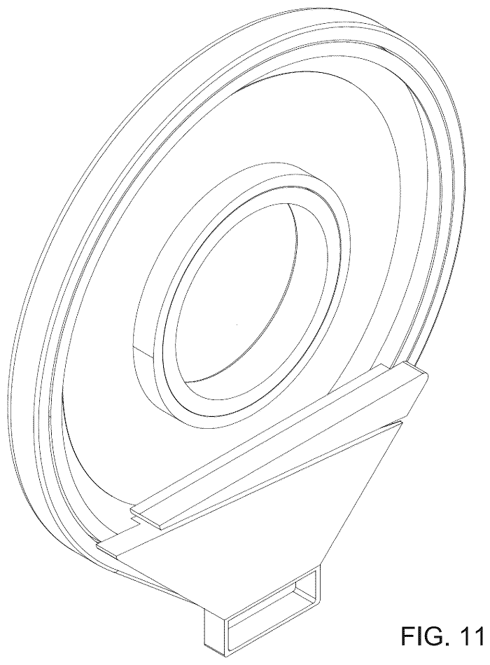

FIG. 11 is an upside down perspective view of the cartridge of FIG. 1. Only major differences or points of emphasis are described herein. In particular, this view shows one representation of a notch (2) on the bottom of the feeder (1). Moreover, this view also shows one representation of a space between the feeder (1) and the inner disk (5) for an assembled Cartridge. In one aspect, the notch (2) helps a user with placing a pre-coiled wire/catheter into the Cartridge (e.g., through the space between the feeder (1) and the inner disk (5)) and with fishing an end of the inserted pre-coiled wire/catheter into the Cartridge, and with threading the end through the guide wire/catheter guide (12) and into/through the feeder (1). In another aspect, the space between the feeder (1) and the inner disk (5) effectively allows for loading of the assembled Cartridge with wire/catheter without having to remove the feeder (1). Of course, in another aspect, in another method of loading the Cartridge, the feeder (1) may be removed to allow for insertion of a pre-coiled wire/catheter into the Cartridge.

FIG. 12 is a first sectional view of a reel cartridge with housing. Only major differences or points of emphasis are described herein. In particular, this view shows how the entire cartridge assembly fits within a slot from a vertical position and how each cartridge is flush to the walls of the housing leading to the next slot. This influences the solution to move more laterally rather than vertically in the slot.

FIG. 13 is a second sectional view of the reel cartridge with housing of FIG. 12. Only major differences or points of emphasis are described herein. In particular, this view shows the position of the cartridge assembly within a slot from lateral position. This view also shows the walls of the housing to be solid so that each “bath” is individual and utilizes the proper amount of solution. Along with the proper amount of solution this also helps with increasing contact with the solution further helping with the cleaning process. A gap from the bottom of the housing to the bottom of the cartridge should be seen as a minimum point for fluid used in the slot. The solution must at least be as high as the most bottom part of the inner disk to be effective. However, due to displacement of fluids, solution is advised to not be more than ½ full to prevent spillages.

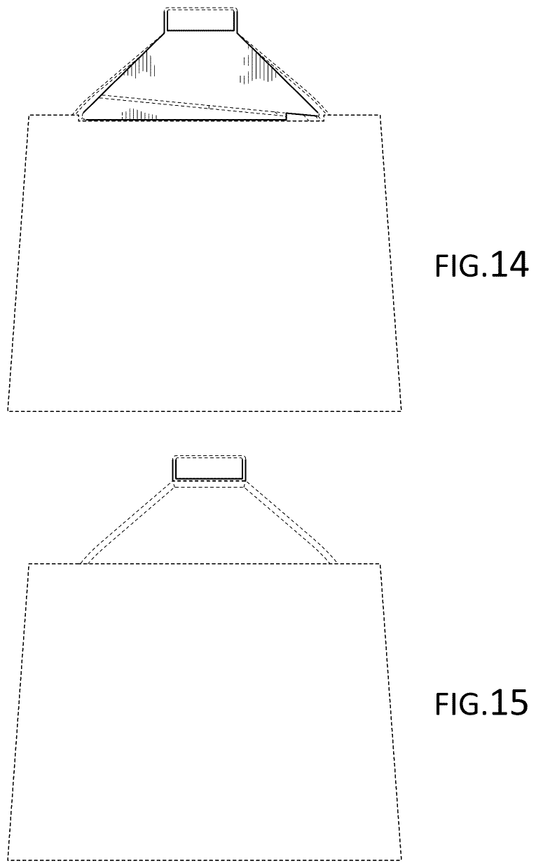

FIG. 14 is a front view of another embodiment of a reel cartridge with housing.

FIG. 15 is a rear view thereof.

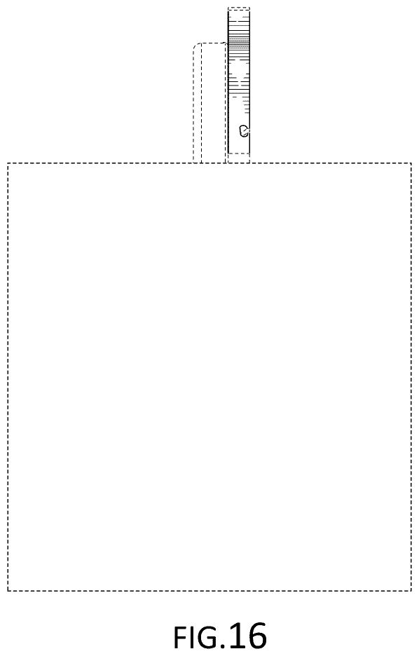

FIG. 16 is a left side view thereof.

FIG. 17 is a right side view thereof.

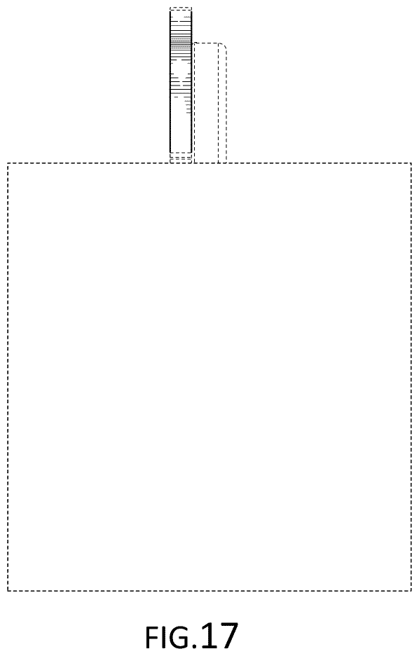

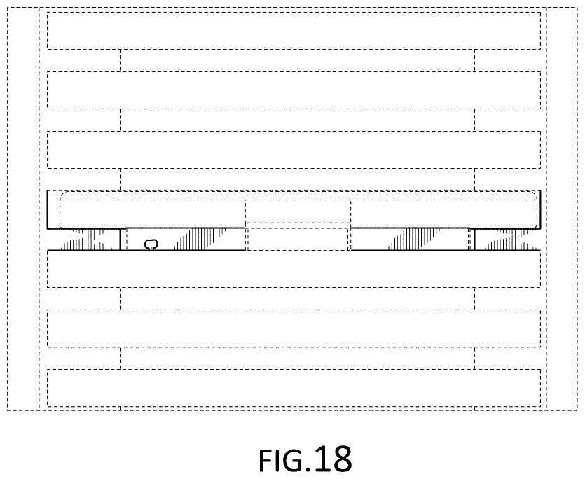

FIG. 18 is a top view thereof.

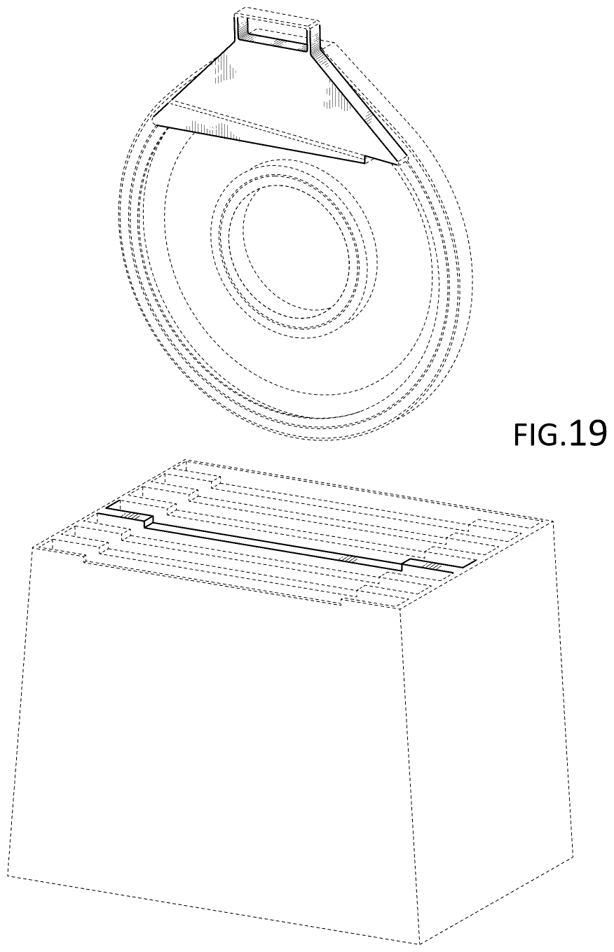

FIG. 19 is a first side exploded perspective view thereof.

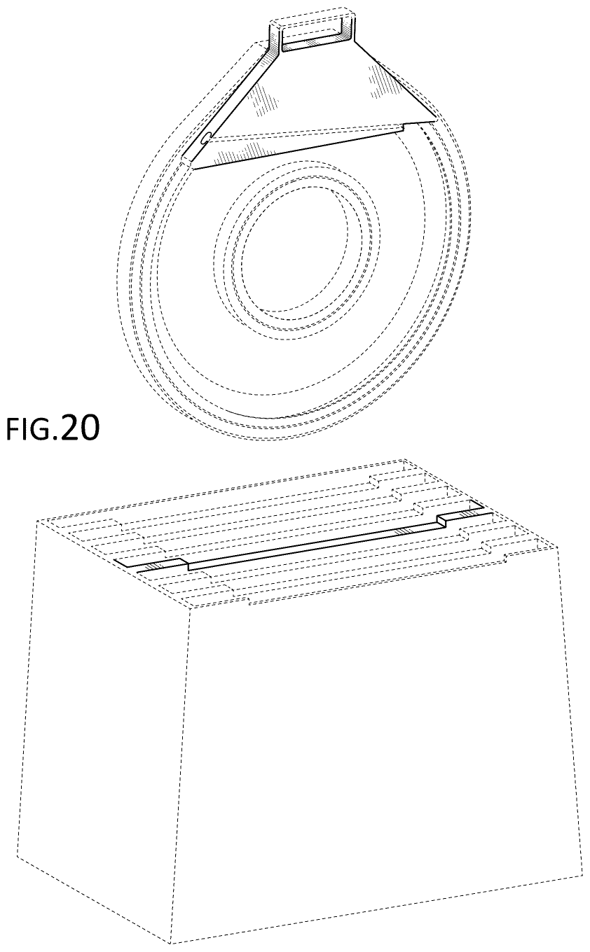

FIG. 20 is a second side exploded perspective view thereof.

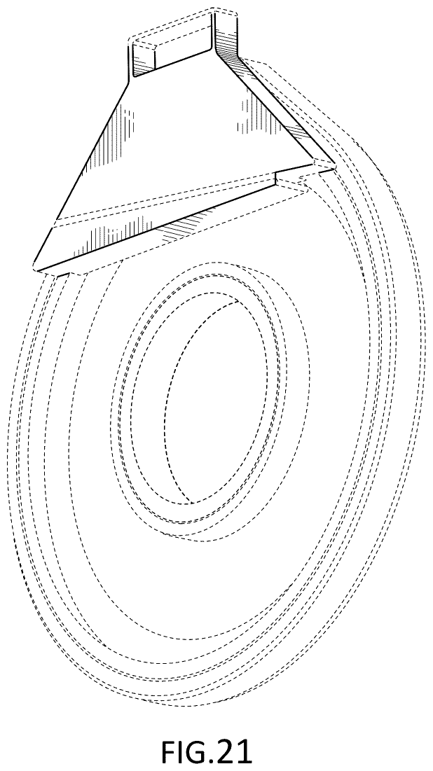

FIG. 21 is a first side perspective view of another embodiment of a reel cartridge.

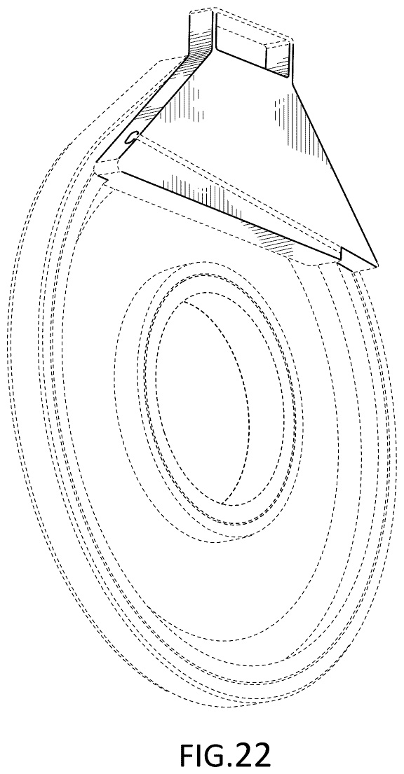

FIG. 22 is a second side perspective view thereof.

FIG. 23 is a front view thereof.

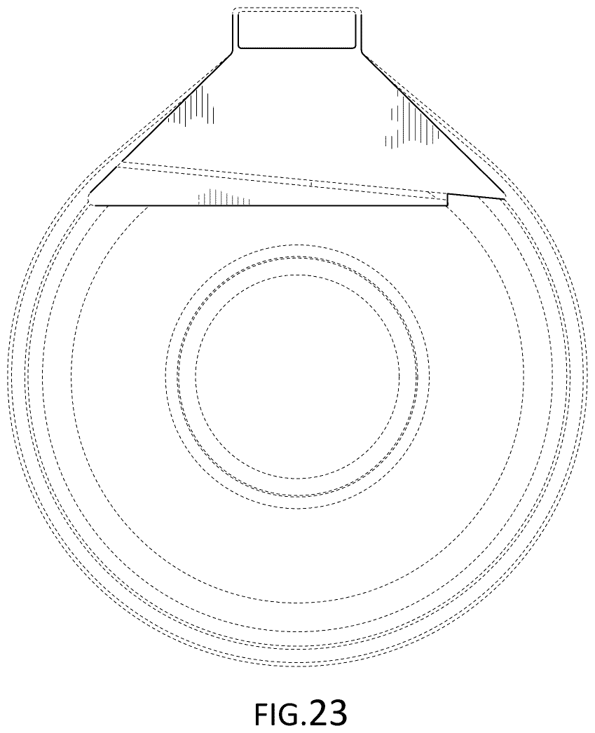

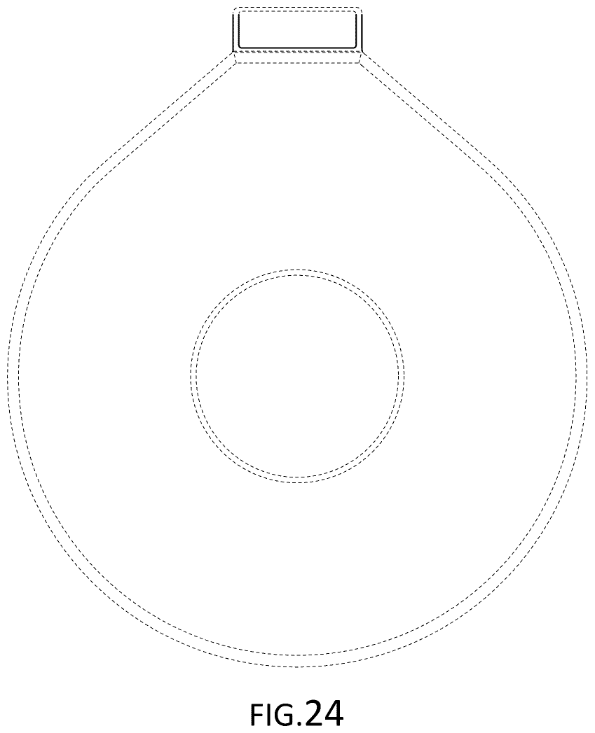

FIG. 24 is a rear view thereof.

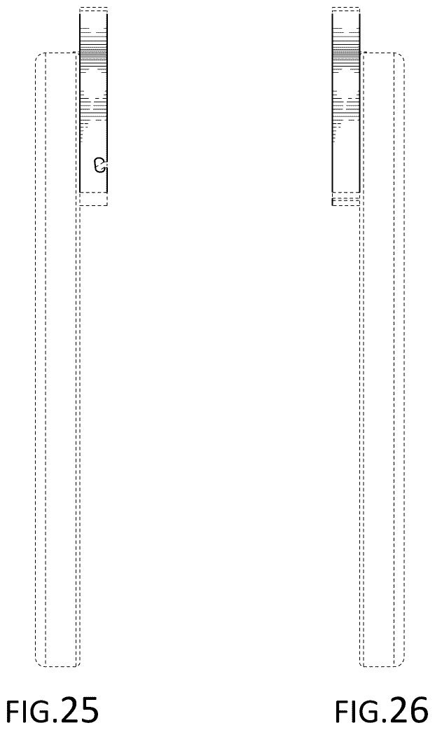

FIG. 25 is a left side view thereof.

FIG. 26 is a right side view thereof.

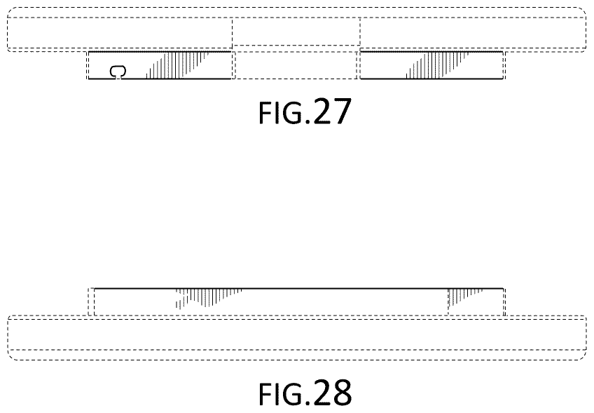

FIG. 27 is a top view thereof.

FIG. 28 is a bottom view thereof.

FIG. 29 is a first side perspective view of another embodiment of a reel cartridge.

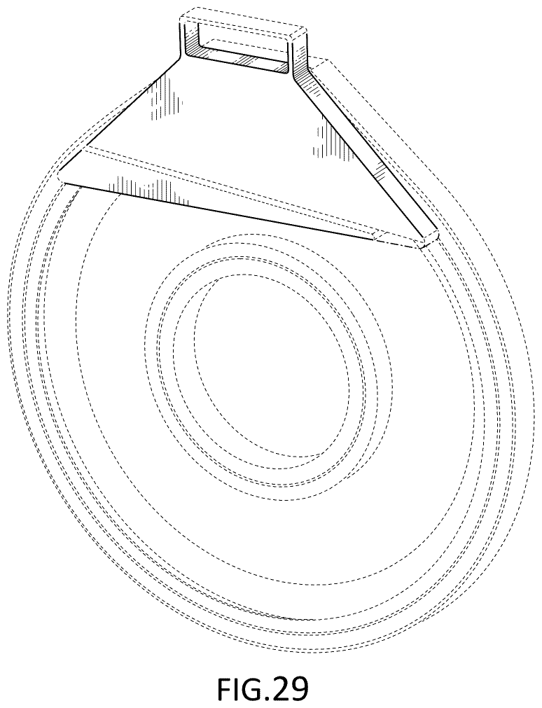

FIG. 30 is a second side perspective view thereof.

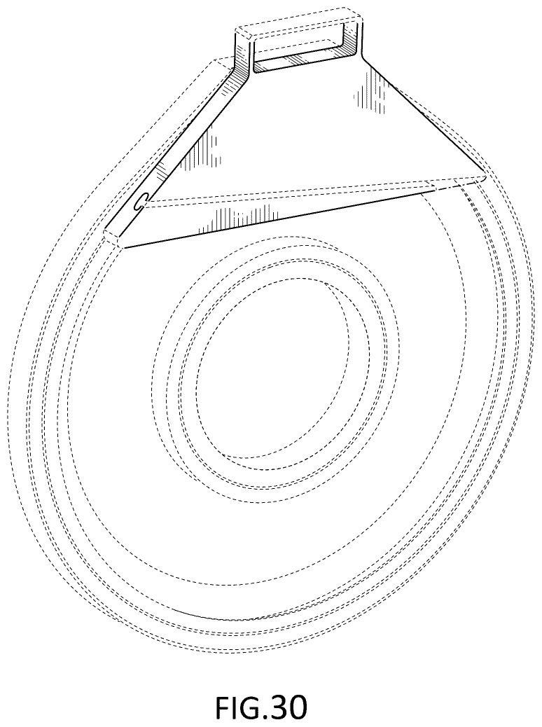

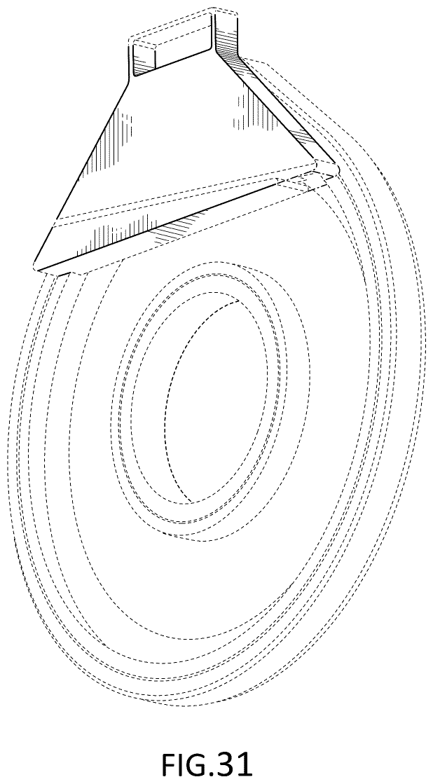

FIG. 31 is a third side perspective view thereof.

FIG. 32 is a fourth side perspective view thereof.

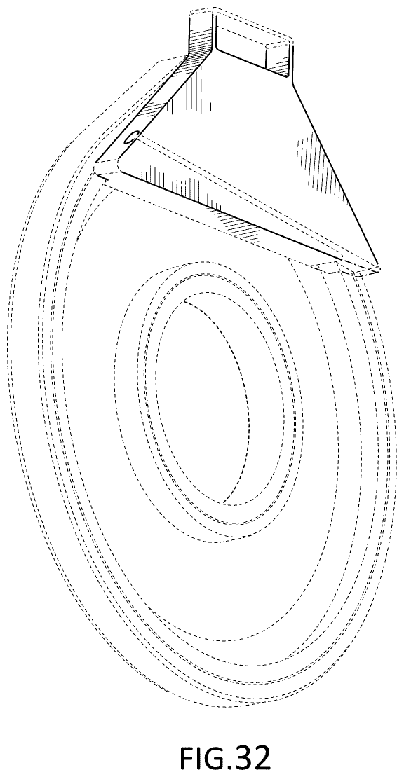

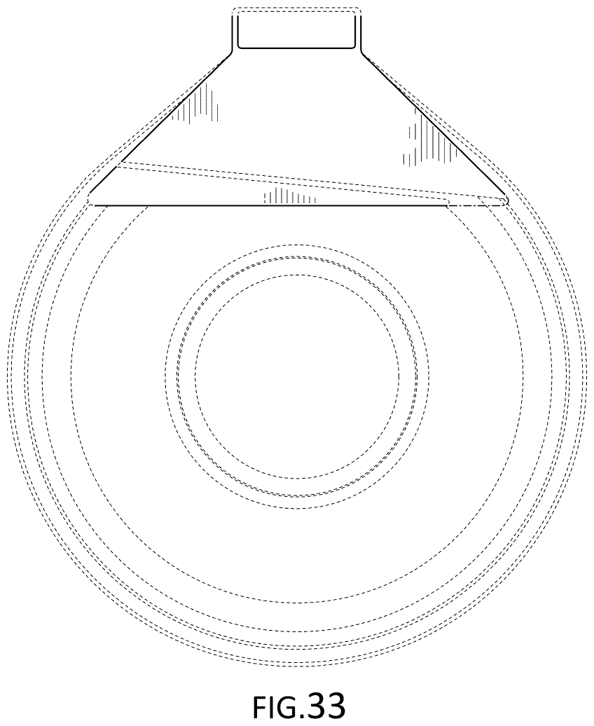

FIG. 33 is a front view thereof.

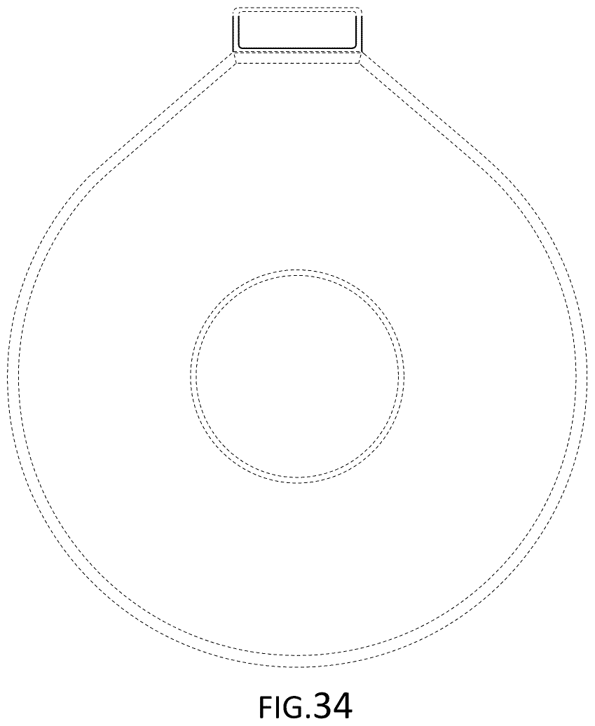

FIG. 34 is a rear view thereof.

FIG. 35 is a left side view thereof.

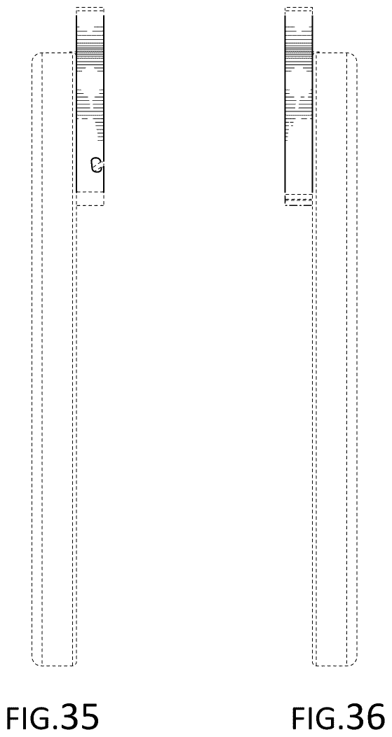

FIG. 36 is a right side view thereof.

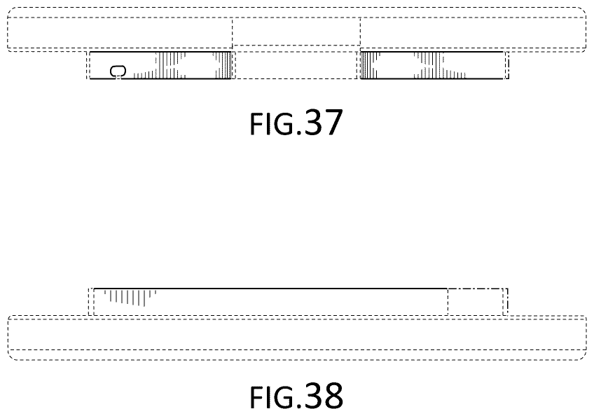

FIG. 37 is a top view thereof.

FIG. 38 is a bottom view thereof.

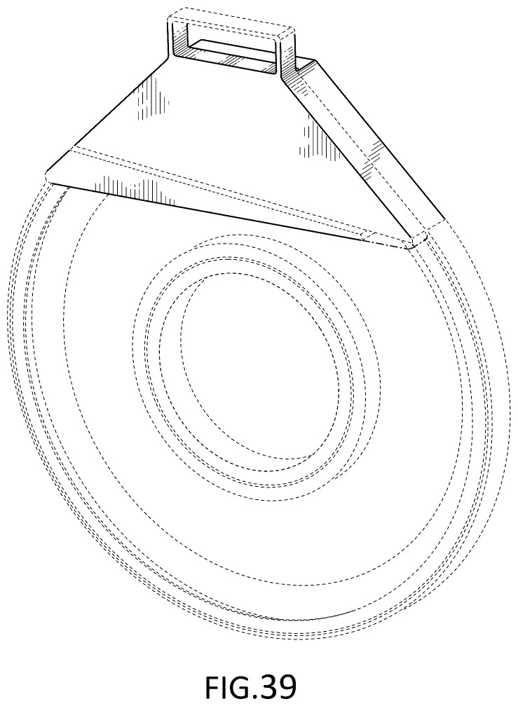

FIG. 39 is a first side perspective view of another embodiment of a reel cartridge.

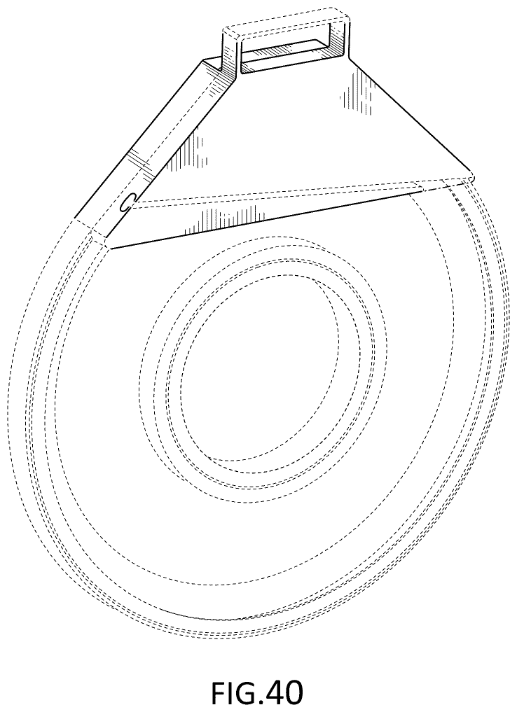

FIG. 40 is a second side perspective view thereof.

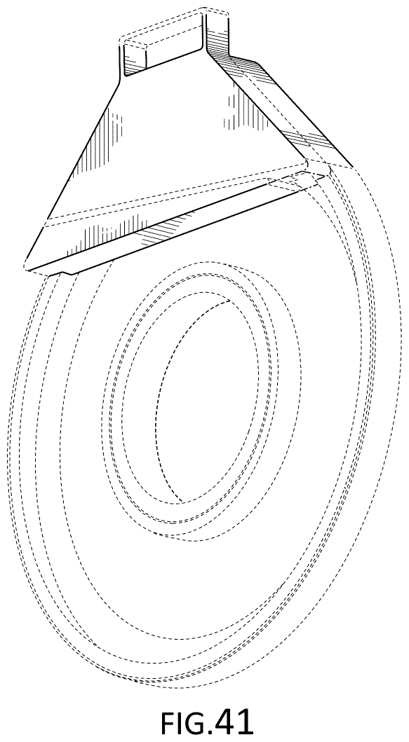

FIG. 41 is a third side perspective view thereof.

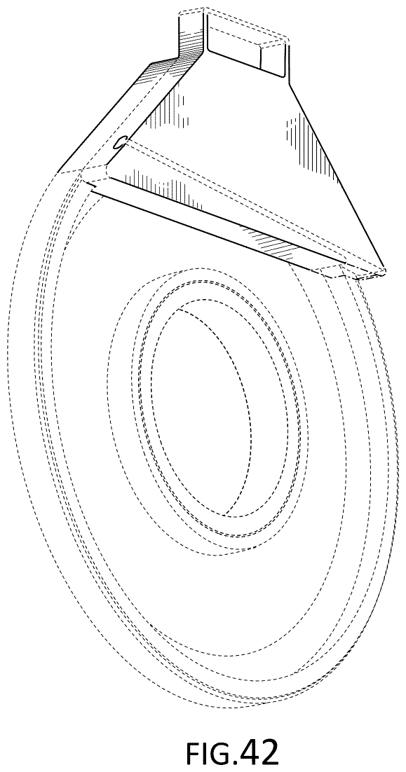

FIG. 42 is a fourth side perspective view thereof.

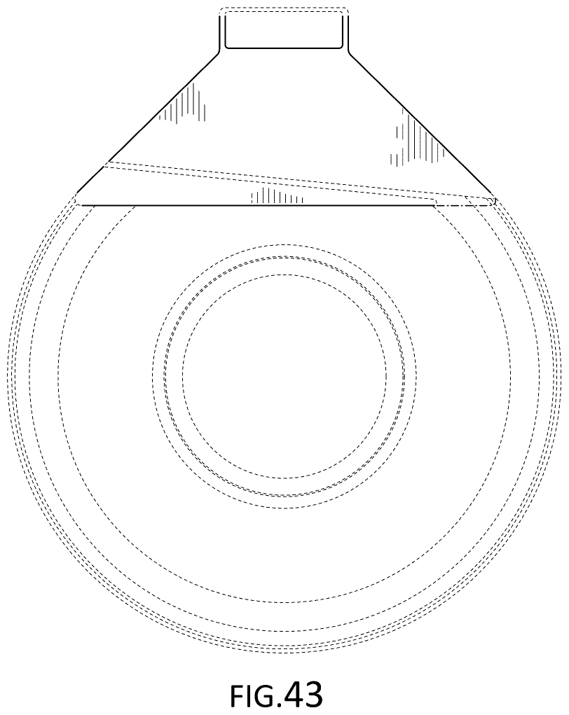

FIG. 43 is a front view thereof.

FIG. 44 is a rear view thereof.

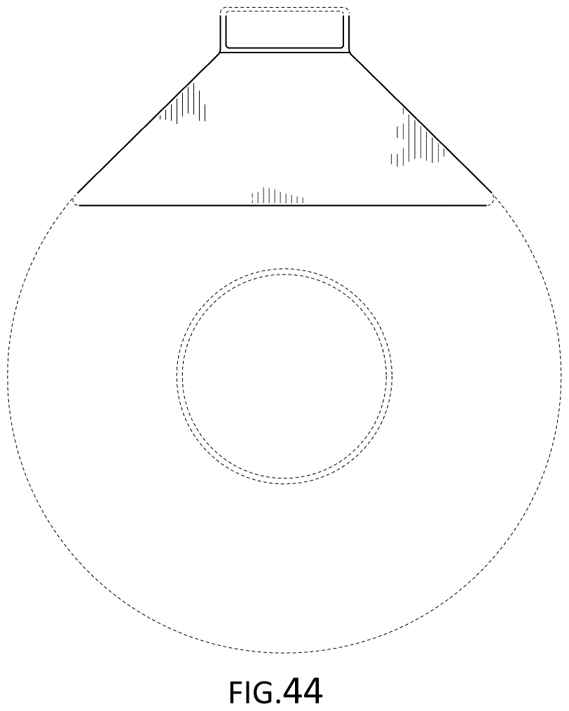

FIG. 45 is a left side view thereof.

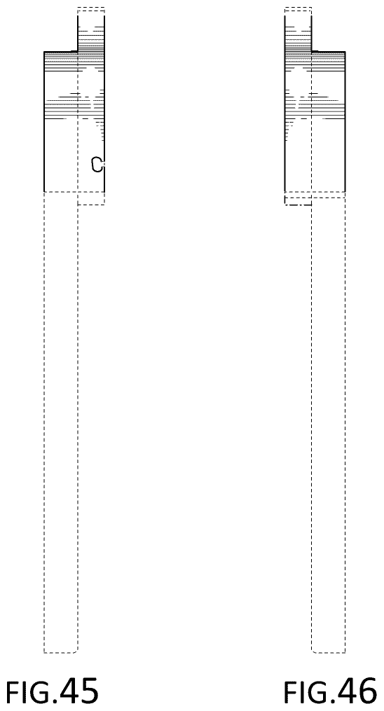

FIG. 46 is a right side view thereof.

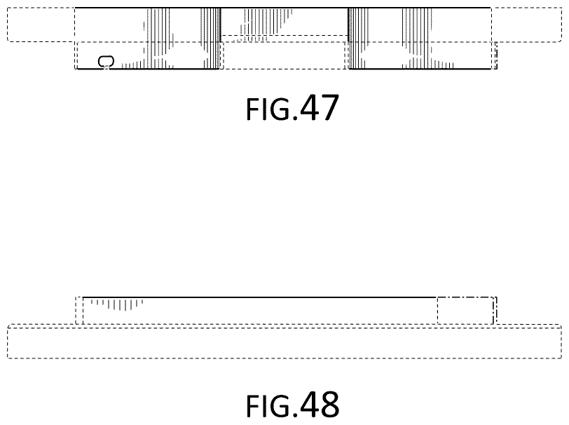

FIG. 47 is a top view thereof.

FIG. 48 is a bottom view thereof.

The bottom view of a reel cartridge with housing is flat and unornamented.

The bottom view of a housing is flat and unornamented.

In the specified embodiments, the claimed design extends to the boundary lines but does not include the boundary lines. In another embodiment, the claimed design extends to the boundary lines and does include the boundary lines.

### EMBODIMENTS

Certain implementations of systems and methods consistent with the present disclosure are provided as clauses

Clause 1. An organization system, comprising: a slotted housing comprising a top notch; and a cartridge configured to receive a spooled elongated profile object, and configured to be received by a slot of the slotted housing; wherein, the top notch is defined by a housing wall; wherein the slot and the housing wall form a soaking bath for the cartridge and the spooled elongated profile object therein; and wherein, when the spooled elongated profile object is unspooled from the cartridge, the top notch limits the cartridge from rocking or rotating within the slot.

Clause 2. The organization system of clause 1, wherein the housing wall defines the top notch.

Clause 3. The organization system of clause 1, comprising a plurality of cartridges, wherein the slotted housing comprises a plurality of housing walls defining a plurality of soaking baths, each soaking bath having a corresponding cartridge.

Clause 4. The organization system of clause 3, wherein each housing wall defines a corresponding top notch.

Clause 5. The organization system of clause 1, wherein the spooled elongated profile object is a wire for surgical, endovascular, radiology, or other invasive or partially invasive procedures.

Clause 6. The organization system of clause 1, wherein the spooled elongated profile object is a catheter for surgical, endovascular, radiology, or other invasive or partially invasive procedures.

Clause 7. The organization system of clause 1, wherein the spooled elongated profile object is a wire for surgical, endovascular, radiology, or other invasive or partially invasive procedures.

Clause 8. The organization system of clause 1, wherein the cartridge comprises a port for receiving the spooled elongated profile object.

Clause 9. The organization system of clause 8, wherein the port is for dispensing the spooled elongated profile object.

Clause 10. An organization system, comprising: a slotted housing; and a cartridge configured to receive a spooled elongated profile object, and configured to be received by a slot of the slotted housing, the cartridge comprising: an outer disk; an inner rotating disk; and a feeder; wherein, when the spooled elongated profile object is unspooled from the cartridge, the slotted housing limits the cartridge from rocking or rotating within the slot.

Clause 11. The organization system of clause 10, wherein the inner rotating disk rotates inside of the outer disk to receive or dispense the spooled elongated profile object.

Clause 12. The organization system of clause 10, wherein the feeder comprises a handle portion.

Clause 13. The organization system of clause 10, wherein the slotted housing comprises a housing wall defining a top notch, wherein the slot and the housing wall form a soaking bath for the cartridge and the spooled elongated profile object therein.

Clause 14. The organization system of clause 10, comprising a plurality of cartridges, wherein the slotted housing comprises a plurality of housing walls defining a plurality of soaking baths, each soaking bath having a corresponding cartridge.

Clause 15. The organization system of clause 14, wherein each housing wall defines a corresponding top notch.

Clause 16. The organization system of clause 10, wherein the spooled elongated profile object is a wire for surgical, endovascular, radiology, or other invasive or partially invasive procedures.

Clause 17. The organization system of clause 10, wherein the spooled elongated profile object is a catheter for surgical, endovascular, radiology, or other invasive or partially invasive procedures.

Clause 18. The organization system of clause 10, wherein the spooled elongated profile object is a wire for surgical, endovascular, radiology, or other invasive or partially invasive procedures.

Clause 19. The organization system of clause 10, wherein the cartridge comprises a port for receiving the spooled elongated profile object.

Clause 20. The organization system of clause 19, wherein the port is for dispensing the spooled elongated profile object.

Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

Therefore, the following is claimed:

1. An organization system, comprising:
a slotted housing comprising a top notch; and
a cartridge configured to receive a spooled elongated profile object, and configured to be received by a slot of the slotted housing;
wherein, the top notch is defined by a housing wall;
wherein the slot and the housing wall form a soaking bath for the cartridge and the spooled elongated profile object therein; and
wherein, when the spooled elongated profile object is unspooled from the cartridge, the top notch limits the cartridge from rocking or rotating within the slot.

2. The organization system of claim 1, wherein the housing wall defines the top notch.

3. The organization system of claim 1, comprising a plurality of cartridges, wherein the slotted housing comprises a plurality of housing walls defining a plurality of soaking baths, each soaking bath having a corresponding cartridge.

4. The organization system of claim 3, wherein each housing wall defines a corresponding top notch.

5. The organization system of claim 1, wherein the spooled elongated profile object is a wire for surgical, endovascular, radiology, or other invasive or partially invasive procedures.

6. The organization system of claim 1, wherein the spooled elongated profile object is a catheter for surgical, endovascular, radiology, or other invasive or partially invasive procedures.

7. The organization system of claim 1, wherein the spooled elongated profile object is a wire for surgical, endovascular, radiology, or other invasive or partially invasive procedures.

8. The organization system of claim 1, wherein the cartridge comprises a port for receiving the spooled elongated profile object.

9. The organization system of claim 8, wherein the port is for dispensing the spooled elongated profile object.

10. An organization system, comprising:
a slotted housing; and
a cartridge configured to receive a spooled elongated profile object, and configured to be received by a slot of the slotted housing, the cartridge comprising:
an outer disk;
an inner rotating disk; and
a feeder;
wherein, when the spooled elongated profile object is unspooled from the cartridge, the slotted housing limits the cartridge from rocking or rotating within the slot.

11. The organization system of claim 10, wherein the inner rotating disk rotates inside of the outer disk to receive or dispense the spooled elongated profile object.

12. The organization system of claim 10, wherein the feeder comprises a handle portion.

13. The organization system of claim 10, wherein the slotted housing comprises a housing wall defining a top notch, wherein the slot and the housing wall form a soaking bath for the cartridge and the spooled elongated profile object therein.

14. The organization system of claim 10, comprising a plurality of cartridges, wherein the slotted housing comprises a plurality of housing walls defining a plurality of soaking baths, each soaking bath having a corresponding cartridge.

15. The organization system of claim 14, wherein each housing wall defines a corresponding top notch.

16. The organization system of claim 10, wherein the spooled elongated profile object is a wire for surgical, endovascular, radiology, or other invasive or partially invasive procedures.

17. The organization system of claim 10, wherein the spooled elongated profile object is a catheter for surgical, endovascular, radiology, or other invasive or partially invasive procedures.

18. The organization system of claim 10, wherein the spooled elongated profile object is a wire for surgical, endovascular, radiology, or other invasive or partially invasive procedures.

19. The organization system of claim 10, wherein the cartridge comprises a port for receiving the spooled elongated profile object.

20. The organization system of claim 19, wherein the port is for dispensing the spooled elongated profile object.
